(19) United States
(12) Patent Application Publication
(10) Pub. No.: US 2005/0211243 A1
(43) Pub. Date: Sep. 29, 2005

(54) Inhaler

(76) Inventor: Ralf Esser, Hennef (DE)

Correspondence Address: Glenn E Klepac, 825 Fifth Avenue, Suite 209, New Kensington, PA, 15068-6310, US

(21) Appl. No.: 10/514,157

(22) PCT Filed: May 13, 2002
(86) PCT No.: PCT/DE02/01712

§ 371 Date: Nov. 12, 2004

(51) Int. Cl. A61M 15/00; A61M 16/10

(52) U.S. Cl. 128/203.12

(57) ABSTRACT

The invention relates to an inhaler including a catalytic burner, a fuel tank (6) connected to the burner, a container (15) for inhalation additives such as aromatic substances and/or active ingredients, at least one inlet (2) for a gaseous mixture containing oxygen (in particular for air), and an outlet (3) for an inhalation mixture containing aromatic substances and/or active ingredients. The fuel tank (6) preferably contains hydrogen.

Inhalers are used mostly for medicinal or therapeutic purposes and are designed differently depending on their application.

There are inhalers for smoking cessation that consist of a mouth piece and an end piece and that have an air channel into which a nicotine capsule can be inserted. Through the air stream produced by “puffing” on the mouth piece, nicotine is released in the nicotine capsule. Contrary to cigarette or cigar smoking, such inhalers have the advantage that their use does not affect the indoor air quality and thus the well-being of others. They are therefore particularly well suited for use in non-smoking areas, for example on an airplane. This type of inhaler, however, has the disadvantage that the released nicotine still adversely affects the health of the smoker. Moreover, the smoking sensation experienced when puffing on an inhaler is hardly comparable to that of smoking cigarettes or cigars, as the inhaled air is generally cold and no feeling of gratification ensues due to the lack of smoke.

Other inhalers for the medicinal treatment of respiratory diseases or colds have a heatable water container to which a nose and mouth piece is attached. Essential oils or pharmaceutical active ingredients can be added to the water in the water container so that when the water is heated, these can be inhaled together with the water vapor. These types of inhalers use a heating spiral as heat source, which can be powered with alternating current from “the wall outlet” or with direct current, for example, from a car battery. They have the disadvantage of being bulky. Moreover, due to the required power supply, they cannot be used everywhere.

These disadvantages also exist with inhalers that produce aerosols to be inhaled from solutions that contain pharmaceutical active ingredients. In particular, two types of devices are used for the production of aerosols—ultrasonic nebulizers and pneumatic nebulizers. Ultrasonic nebulizers atomize the solution via a membrane that is brought to vibration by ultrasonic waves; in pneumatic nebulization the solution flows through a nozzle under pressure.

In comparison, the object of the present invention is to create a new type of inhaler that is suitable for the aforementioned applications but does not have the above described disadvantages.

This object is achieved by an inhaler comprising a catalytic burner and a fuel container containing, in particular, hydrogen which is connected to said burner, a container for inhalation additives such as aromatic substances and/or active ingredients, at least one inlet for oxygen or a gaseous mixture containing oxygen, in particular for air, and an outlet for an inhalation mixture containing aromatic substances and/or active ingredients.

One basic thought underlying the invention is to use not only the electric energy released during the catalytic combustion of the hydrogen, but also the resulting waste gases, to produce the inhalation mixture containing aromatic substances and/or active ingredients. Thus, the heat present in the waste gases can be used to heat the inhalation additives, for example by heating the container containing the inhalation additives. The waste gas heat can be also used to heat the inhalation mixture, which leads to the advantage of a higher capacity for the absorption of water vapor, so that the active ingredients can be transported in higher concentrations than with a cold inhalation mixture. At the same time, the waste gases can be added directly to the inhalation mixture because they are generally absolutely free of harmful substances, for example when burning hydrogen.

This results in a number of advantages. Thus, an inhaler supplied with energy via the catalytic burner is independent from external energy sources. The required energy is released in an non-polluting way, in particular with the catalytic combustion of hydrogen, as the combustion product is nothing but harmless water vapor, which is even used for the transport of aromatic substances and active ingredients. The fuel that is being used is thus not only an energy supplier, but it also supplies a means to transport the aromatic substances or active ingredients. But even other fuels, such as natural gas, for instance, could be used, as the flameless catalytic combustion, due to the much lower combustion temperature, only generates water vapor and CO₂ as waste gases, which can be admixed to the inhalation mixture without any concerns.

In principle, the catalytic converter according to the invention can be designed such that when the inhaler is in operation, the warm waste gases, possibly in combination with ambient air, are led through the container containing the inhalation substances, with the waste gas flow absorbing the inhalation additives. Depending on the type of inhalation additives [or] active ingredients, this can be provided in liquid, but also in solid, powdery form. If the inhalation additives are liquid, they can evaporate at their surface to the waste gas. If the inhalation additives are solid, they can, with adequate waste gas flow conduction and flow speed, be entrained by the waste gases so that the inhalation mixture is an aerosol containing solid particles.

Due to the easily controlled reaction course, a fuel cell lends itself as a catalytic burner. Modem fuel cells are highly efficient and can be designed such that the quantity of air blowing by the fuel cell's catalytic membrane automatically regulates the quantity of hydrogen ions passing through it.

In an advantageous development of one such fuel cell used as a catalytic burner, the fuel cell is wound in a coil-like fashion. Coiling the fuel cell not only reduces the space required by it considerably, but also automatically creates and air channel through which the oxygen or the oxygen-containing gaseous mixture can be conducted.

Compared to the dosing of powdery inhalation additives, the dosing of inhalation additives dissolved in water or other carriers is simpler and thus preferred, as there are no or only minor requirements with regard to the waste gas flow conduction and speed. Depending on the design configuration of the inhaler, another advantage can be that, subject to a suitable concentration of dissolved inhalation additives, no further control of the addition of these additives to the inhalation mixture is required.

The inhaler according to the invention can preferably comprise a heating device powered by the catalytic burner, that will cause the solution containing the inhalation additives to evaporate, so that the inhalation additives are contained as vapor in the inhalation mixture. Such a heating device could, for example, be a heating spiral within the container for the dissolved inhalation additives.

It is, however, also conceivable to provide a heat exchanger through which the heat of the waste gases is transferred to the solution.

Moreover, the inhaler can comprise a nebulizer powered by the catalytic burner, in particular an ultrasonic or a pneumatic nebulizer for the conversion of the solution into an aerosol. Two principles can basically be implemented to nebulize the solution using pressure. The pressure in the vessel containing the solution with the inhalation additives required for the pneumatic nebulization can be generated either by heating the solution, or, preferably, through a compressor. It is, however, also conceivable to use solvent additives with a relatively low boiling point, compared to water, to generate pressure so that sufficient pressure can be built up in the vessel using only a little heat. This makes it possible to regulate the pressure and thus the nebulization through a controlled supply of heat.

In another advantageous embodiment, the inhaler comprises an additional water tank as well as a heating device powered by the catalytic burner for the evaporation of the water. In this case, the entire inhalation mixture need not be created in the catalytic burner. Rather, the heat released in the hydrogen combustion can be used to heat the water to water vapor, which is then added to the water vapor exiting from the catalytic burner.

In yet another preferred embodiment, the inhaler comprises a control system for the dosing of the addition of aromatic substances or active ingredients to the inhalation mixture.

It is also an advantage if the fuel container and all other containers are replaceable and/or refillable. This allows for the inhaler to be used for a long time.

Furthermore, it can be advantageous if a mixing device for the mixing of the inhalation mixture with ambient air is located upstream of the outlet. This allows the user to dose the quantity of the inhalation mixture to be inhaled as needed.

Preferably, the inhaler according to the invention should be provided with a control system to allow the oxygen volume led through the fuel cell to be regulated. This affords an advantage in particular in cases where the inhaler is to generate a time-constant amount of the inhalation mixture, in which case the functionality of the inhaler is equivalent to that of a traditional inhaler for medicinal purposes.

Moreover, an inhaler according to the invention may comprise a mouth piece. Such a mouth piece may, for instance, be shaped in the form of a mouth piece for cigarettes, and is particularly well suited for inhalers with which the inhalation mixture is inhaled solely through the mouth. Instead of a mouth piece, it is, however, also possible to provide a mask at the outlet of the inhaler, with which mouth and nose of a user can be covered.

The current generated in the catalytic combustion, in particular in a fuel cell, can also be utilized. On one hand, it can be used for the operation of a heating spiral to heat up the additives or water. It can, however, also be used for a lamp unit, which can, for example, be used to indicate when the inhaler is in use. On the other hand, it can also be used to imitate the glow of a cigarette or a cigar, if the inhaler is used as a cigarette substitute in smoking cessation or as a new form of stimulant.

What all embodiments of the invention have in common is that the inhaler can be used irrespective of location and availability of an external power source, that a warm inhalation mixture can be produced that is perceived by the inhaling person as pleasant, and that not only the [. . . ]from the catalytic burner, but also the combustion products and their waste heat can be used, if necessary, for the preparation of the inhalation mixture.

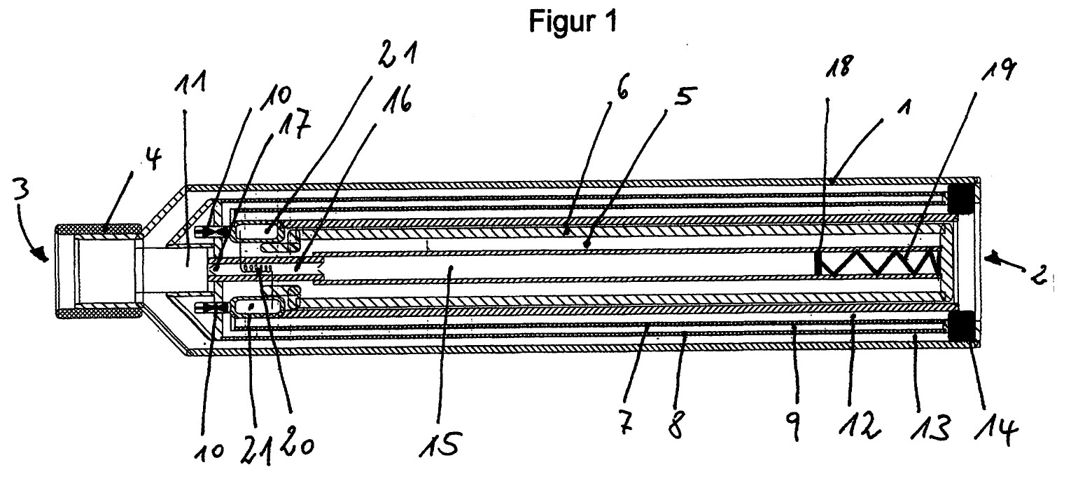

In the following, the present invention will be explained in greater detail on the basis of FIG. 1, which illustrates the operating principle of an example of an inhaler.

The inhaler shown in principle in FIG. 1, comprises an oblong, cylindrical hollow body 1 with and inlet 2 for air and an outlet 3 for an inhalation mixture. A replaceable mouth piece 4 is provided at the outlet 3. On its inside, the hollow body 1 comprises a concentrically arranged wall 5, in which an essentially cylindrical hydrogen-containing fuel container 6 is located, whose bottom is facing the air intake 2 and whose gas outlet is facing the outlet 3.

An inner membrane 7 and an outer membrane 8, both of which form a fuel cell, are arranged concentrically and spaced out between the wall 5 and the interior wall of the hollow body. The hollow space 9 enclosed between the two membranes 7, 8 is closed off with a gastight seal on the side that is facing the inlet 2. On the side that is facing the outlet 3, the hollow space 9 is coupled to the gas outlet at the fuel container 6, with the possibility to control or interrupt the hydrogen flow flowing from the fuel container 6 to the hollow space 9 through valves 10.

The channels 12, 13 resulting between the interior wall of the hollow body and the outer membrane 8 as well as between the inner membrane 7 and the walls are connected, at their end facing the inlet 2, to the inlet 2 via a ring-shaped air filter 14. Their opposite ends open into a mixing chamber 11 arranged upstream in front of the outlet 3 (the connection between the channel 12, formed by the inner membrane 7 and the wall 5, and the mixing chamber 11 is not shown).

Concentrically placed inside the fuel container 6, a cylindrical active ingredient container 15 is provided for inhalation additives that are dissolved in a fluid, for example in water. The bottom of the active ingredient container 15 is firmly anchored to the bottom of the fuel container 6. As outlet for the active ingredients, a hollow needle 16 is provided, which passes through the gas outlet of the fuel container 6 and opens into the mixing chamber 11. The opening of the hollow needle 16 facing the mixing chamber 11 is provided with a pressure relief valve 17. Inside the active ingredient container 15, a piston 18 is provided, which is pushed towards the hollow needle 16 by a spring 19 resting on the bottom of the active ingredient container 15. This pressure pushes the solution to which the inhalation additives have been admixed into the hollow needle 16.

A spiral-wound filament 20 is arranged inside the hollow needle 16 in order to evaporate the fluid containing the inhalation substances. The spiral-wound filament 20 can be supplied with current via the membranes 7, 8 of the fuel cell, as can additional components, such as, for example, a compressor for pneumatic nebulization or an ultrasonic vaporizer.

Moreover, the fuel cell supplies two rechargeable batteries 21 located in the wall 5 with current. Powered via the batteries 18 or directly from the current produced by the fuel cell, a control system not shown here for controlling the hydrogen supply via the valves 10 can be provided. In addition, a connection for other consumers can be provided, which is supplied with current either directly from the fuel cell or via the battery (connection not shown here).

In order to inhale, the inhaler's mouth piece 4 is placed to the mouth and air is sucked into the inhaler via the inlet 2. In the process, the sucked-in air flows past the membranes 7, 8 and reacts on the membranes 7, 8 with the hydrogen, so that water vapor is generated and an air/water vapor mix results. At the same time, the current produced by the fuel cell in that reaction is conducted to the spiral-wound filament, so that the fluid located in the hollow needle and containing the inhalation additives is evaporated. Due to the excess pressure thus created, the pressure relief valve 16 opens so that vapor containing inhalation additives can escape. This vapor is admixed to the air/water vapor mixture in the mixing chamber 11, so that a mixture of air, water vapor and inhalation additives can be breathed in through the mouth piece.

In order to allow for the inhalation device to be used for a long time, it is recommended to connect the fuel container 6 as well as the active ingredient container 14 to the inhaler in such a way that they can be replaced, or to provide a possibility for refilling them. To this effect, the hollow cylinder can, for example, comprise a valve connection piece, into which the fuel container 6, possibly together with the active ingredient container 14, can be screwed in.

The basic idea of an inhaler powered by a catalytic burner can be realized in a number of other design embodiments. For example, there is no compelling need to arrange the membranes, fuel and active ingredient containers as well as air channels concentrically to each other.

Likewise, additional adjustment and control mechanisms can be provided. While the fuel cells available today are generally self-regulating subject to the supplied air flow, there might be a need to control the hydrogen supply, depending on the area of application. It is also possible, for example depending on the temperature and/or the relative humidity of the air/water vapor/inhalation mixture, to provide an adjustable fresh air supply in the mixing chamber, with which the mixture in the mixing chamber can be cooled. Of course any necessary cooling can also be achieved through a heat exchanger.

List of reference numbers

| | |
|---|---|
| 1 | cylindrical hollow body |
| 2 | inlet |
| 3 | outlet |
| 4 | mouth piece |
| 5 | wall |
| 6 | fuel container |
| 7 | inner membrane |
| 8 | outer membrane |
| 9 | hollow space |
| 10 | valves |
| 11 | mixing chamber |
| 12 | channel |
| 13 | channel |
| 14 | air filter |
| 15 | active ingredient container |
| 16 | hollow needle |
| 17 | pressure relief valve |
| 18 | piston |
| 19 | spring |
| 20 | spiral-wound filament |
| 21 | batteries |

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. Inhaler comprising a catalytic burner and a container (15) for inhalation additives such as aromatic substances and/or active ingredients, at least one inlet (2) for a gaseous mixture containing oxygen, in particular for air, and an outlet (3) for an inhalation mixture containing aromatic substances and/or active ingredients, characterized by a fuel container for containing hydrogen which is connected to the catalytic burner.

16. Inhaler according to claim 15, wherein the catalytic burner is a fuel cell (7, 8).

17. Inhaler according to claim 16, wherein the fuel cell (7, 8) is would in a coil-like fashion.

18. Inhaler according to claim 15, wherein the inhalation additives are dissolved in water or another solvent, said additives and said solvent comprising a solution.

19. Inhaler according to claim 18, wherein the heating device (20) is powered by the catalytic burner for evaporation of the solution containing the inhalation additives.

20. Inhaler according to claim 18, further comprising a nebulizer powered by the catalytic burner, in particular an ultrasonic nebulizer or a pneumatic nebulizer, for nebulization of the solution.

21. Inhaler according to claim 19, further comprising a nebulizer powered by the catalytic burner, in particular an ultrasonic nebulizer or a pneumatic nebulizer, for nebulization of the solution.

22. Inhaler according to claim 15, characterized by an additional water tank.

23. Inhaler according to claim 15, characterized by a control device for the dosing delivery of the inhalation additives to the inhalation mixture.

24. Inhaler according to claim 15, characterized in that the fuel container (6) and all other containers are replaceable and/or refillable.

25. Inhaler according to claim 22, characterized in that the fuel container (6) and all other containers are replaceable and/or refillable.

26. Inhaler according to claim 15, characterized by a mixing device (11) arranged upstream in front of the outlet (3) for addition of ambient air to the inhalation mixture.

27. Inhaler according to claim 15, characterized by a control system for controlling the amount of oxygen led through the fuel cell.

28. Inhaler according to claim 15, further comprising a mouth piece (4).

29. Inhaler according to claim 15, further comprising a lamp unit powered by the catalytic burner.
